(19) United States
(12) Patent Application Publication
(10) Pub. No.: US 2008/0249460 A1
(43) Pub. Date: Oct. 9, 2008

(54) DUAL-SIDED PADDLE FOR USE WITH VIBRATIONAL ENERGY UNDER 1000 HERTZ FOR DERMATOLOGICAL TREATMENT

(75) Inventors: JOHN F. SKINNER, IRVING, TX (US); TERRY OSBORN, ARLINGTON, TX (US)

Correspondence Address: HOWISON & ARNOTT, L.L.P, P.O. BOX 741715, DALLAS, TX, 75374-1715, US

(73) Assignee: VIBRADERM, INC., GRAND PRAIRIE, TX (US)

(21) Appl. No.: 12/101,524

(22) Filed: Apr. 11, 2008

Related U.S. Application Data
(63) Continuation-in-part of application No. 11/472,089, filed on Jun. 21, 2006.
(60) Provisional application No. 60/922,811, filed on Apr. 11, 2007.
(60) Provisional application No. 60/692,443, filed on Jun. 21, 2005.

(51) Int. Cl. A61M 37/00

(52) U.S. Cl. 604/22

(57) ABSTRACT

A treatment paddle is used with a system for dermatological treatment of an individual's skin area. The treatment paddle consists of a first dermatological treatment surface having a sufficiently abrasive side for vibratory removal of an outer skin area of an individual. A second dermatological treatment surface located on an opposite side of the first dermatological treatment surface has a side specifically adapted for the vibratory infusion of a cream, lotion or other dermatological aid into the underlying skin area of said individual. A stem defines first and second ends. The first end of the stem is integrated with the first and second dermatological surfaces and the second end of the stem is adapted for connecting to a vibrational unit to receive vibrational energy therefrom.

### CROSS-REFERENCE TO RELATED APPLICATIONS

This application is a continuation-in-part of U.S. patent application Ser. No. 11/472,089, filed Jun. 21, 2006 entitled “UTILIZING VIBRATIONAL ENERGY UNDER 1000 Hz FOR DERMATOLOGICAL INFUSION TREATMENT MODALITY,” which claims priority to U.S. Provisional Patent Application No. 60/692,443, filed Jun. 21, 2005 entitled “UTILIZING VIBRATIONAL ENERGY UNDER 1000 Hz FOR DERMATOLOGICAL INFUSION TREATMENT MODALITY,” the current application claims benefit to U.S. Provisional Patent Application No. 60/922,811, filed Apr. 11, 2007, entitled “DUAL-SIDED PADDLE FOR USE WITH VIBRATIONAL ENERGY UNDER 1000 HERTZ FOR DERMATOLOGICAL TREATMENT,” all of which are incorporated herein by reference.

### TECHNICAL FIELD

The present invention relates to the use of a single, dual-sided paddle and vibrational energy under 1000 hertz for both dermatological abrasion and substance infusion modalities, and more particularly, but not by way of limitation, to a method of and apparatus for skin rejuvenation incorporating vibrational energy applied to a patient with one of an array of varying sized, dual-sided paddles adapted for both abrasion and infusion.

### BACKGROUND

Dermatological skin treatment processes have long involved some type of exfoliation of the user's skin to remove skin tissue at the surface to remove dead or dry skin and provide access to newer more healthy skin below a minimal surface layer. Additional treatments have involved the use of lotions, creams, etc. for moisturizing and treating various skin infirmities. Some methods of skin rejuvenation have involved the use of an abrasive tape that is applied to the surface of an applicator tip which is adapted for a particular motion. The benefits of exfoliation of a patient's skin and application of dermatological lotions or creams have created a need for the ability to perform both of these processes in a simple to use system that may be implemented by either a doctor or patient.

### SUMMARY

The present invention, as disclosed and described herein, in one aspect thereof, comprises a paddle for use with a system for dermatological treatment of an individual's skin area. The treatment paddle includes a first dermatological treatment surface having a sufficiently abrasive side for vibratory removal of an outer skin area of an individual. A second dermatological treatment surface located on an opposite side of the first dermatological treatment surface has a side specifically adapted for the vibratory infusion of a cream, lotion or other dermatological aid into the underlying skin area of said individual. A stem defines first and second ends. A first end of the stem is integrated with the first and second dermatological surfaces and a second end of the stem is adapted for connection to a vibrational unit to receive vibrational energy therefrom.

### BRIEF DESCRIPTION OF THE DRAWINGS

For a more complete understanding, reference is now made to the following description taken in conjunction with the accompanying Drawings in which:

FIG. 1 is a front elevational view of a dermatological vibrational unit adapted for the select vibratory treatment of a skin surface;

FIG. 2 is a front elevational view of an array of vibratory paddles;

FIG. 3 is a diagrammatical illustration of the utilization of the wand of FIGS. 1 and 2 relative to a patient;

FIG. 4 is an enlarged front elevational view of a non-abrasive paddle constructed with a first type of infusion surface;

FIG. 5 is an enlarged, diagrammatical, side elevational view of the vibratory action of the paddle of FIG. 4 relative to the skin area of a patient for illustrating the infusion angles imparted by the vibrational action of the paddle;

FIG. 6 is a side elevational view of a two sided paddle incorporating a permanent abrasive surface on a first side and a substantially non-abrasive surface on the second, opposite side;

FIG. 7a illustrates three separate paddles having an abrasive side for dermatological treatment;

FIG. 7b is an enlarged view of the abrasive surface of the paddles of FIG. 7a;

FIG. 8a illustrates the opposite less abrasive, infusion side of the paddles of FIG. 7a;

FIG. 8b illustrates a close up view of the non-abrasive infusion surface of the paddles of FIG. 8a;

FIGS. 9a and 9b illustrate the manner in which each side of the dual-sided paddle may be used for skin treatment;

FIG. 10 is a flow diagram describing one embodiment of the dermatological use of the dual-sided paddle.

### DETAILED DESCRIPTION

Referring now to the drawings, wherein like reference numbers are used herein to designate like elements throughout, the various views and embodiments of a dual-sided paddle for use with vibrational energy under 1000 hertz for dermatological treatment is illustrated and described, and other possible embodiments are described. The figures are not necessarily drawn to scale, and in some instances the drawings have been exaggerated and/or simplified in places for illustrative purposes only. One of ordinary skill in the art will appreciate the many possible applications and variations based on the following examples of possible embodiments.

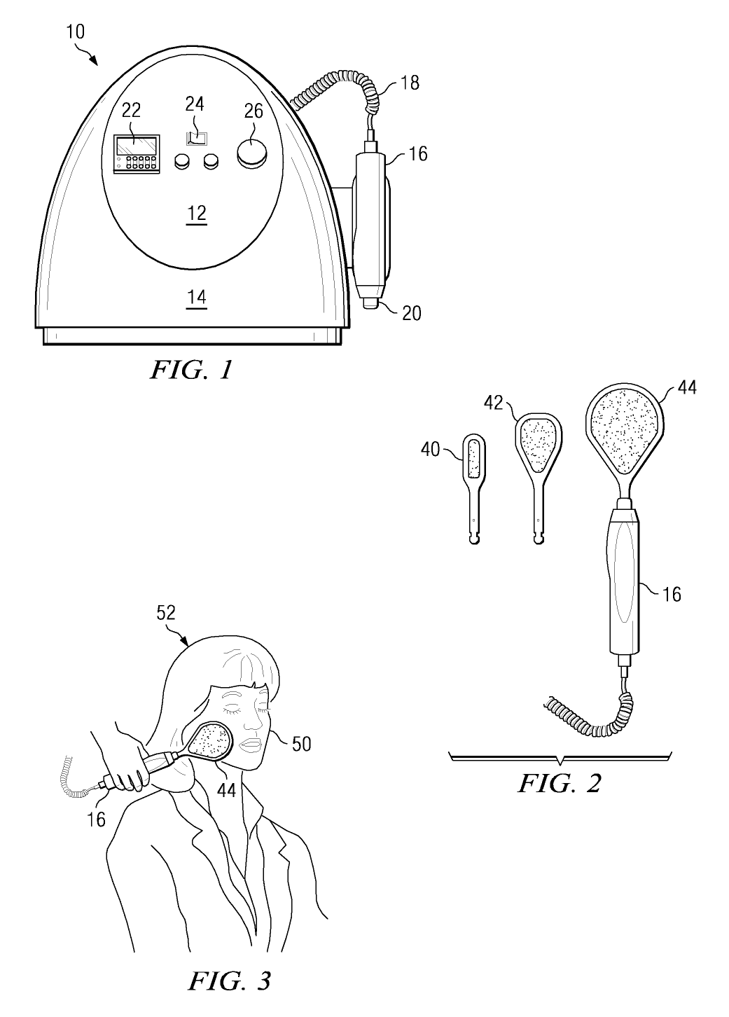

The inventors hereof have found multiple advantages in utilizing dual-sided dermatological treatment paddles in accordance with the principles of the present invention. In that regard, the prior filed patent applications, above referenced, make specific reference to the utilization of a paddle with an abrasive surface on one side for exfoliation purposes and the use of a paddle with a less abrasive surface for substance infusion purposes in the exfoliated treatment area. One referenced embodiment of a paddle design set forth in the prior filed U.S. utility application is a dual-sided paddle wherein a first side includes an abrasive surface and a second, opposite side includes a less abrasive surface adapted for substance infusion. As referenced therein, the designs of the surface of the paddle for infusion were under study and by virtue thereof, the present application provides a refinement for a dual-sided paddle configuration for use with vibrational energy under 1000 Hz and preferably around the range of 80 to 200 Hz, for dermatological treatment. For reference purposes, the following description of FIGS. 1-5 is taken, in part, from the above-referenced full utility application with both the numbers and the substantial description pertaining thereto being left except for select refinements as recognized by the inventors hereof at the time of filing.

Referring now to FIG. 1, the vibrational unit 10 includes a control panel 12 on the frontal portion of a body 14. A wand 16 connected to the body 14 by a cord 18 facilitates the mounting of select dermatological (dermal) rejuvenation equipment, including vibratory paddles described below and in the above-referenced patent applications incorporated herein in their entireties by reference. In one embodiment, a vibrational unit may be incorporated into a portable battery operated device.

Still referring to FIG. 1, the wand 16 has a distal end 20 adapted for receipt of a plurality of paddles therein. Once the selected paddle is inserted, the control panel 12 is utilized to actuate the vibrational energy for a select period of time. In that regard, a monitor 22 may be used relative to a power button 24 and a vibrational energy knob 26 for control of the kinetic energy being imparted to the skin of a patient.

Referring now to FIG. 2, an array of vibratory paddles adapted for dermatological rejuvenation is shown. Paddle 40 is of elongate, unitary construction (that term is used herein to mean a “permanent” assembly with “no removable parts”) providing for vibrational rejuvenation in narrower face regions. Paddle 42 is likewise of wider construction allowing for more select vibrational dermatological applications. Paddle 44 shown mounted into wand 16 is similarly of a larger size adapted for larger more readily available skin areas.

Referring now to FIG. 3, the utilization of the wand 16 and paddle 44 against the face and/or neck 50 of a patient 52 is shown. It may be seen that the vibrational energy imparted to the skin of the patient 52 imparts select energy thereto for the effective removal of outer skin cells from the face region 50. Other areas of the body are, of course, contemplated in accordance with the principles of exclusively dermatological uses of the unit 10. As presently known, use of the unit 10 in the range of 80 to 200 Hz has been shown to be a preferred range in one embodiment of the invention.

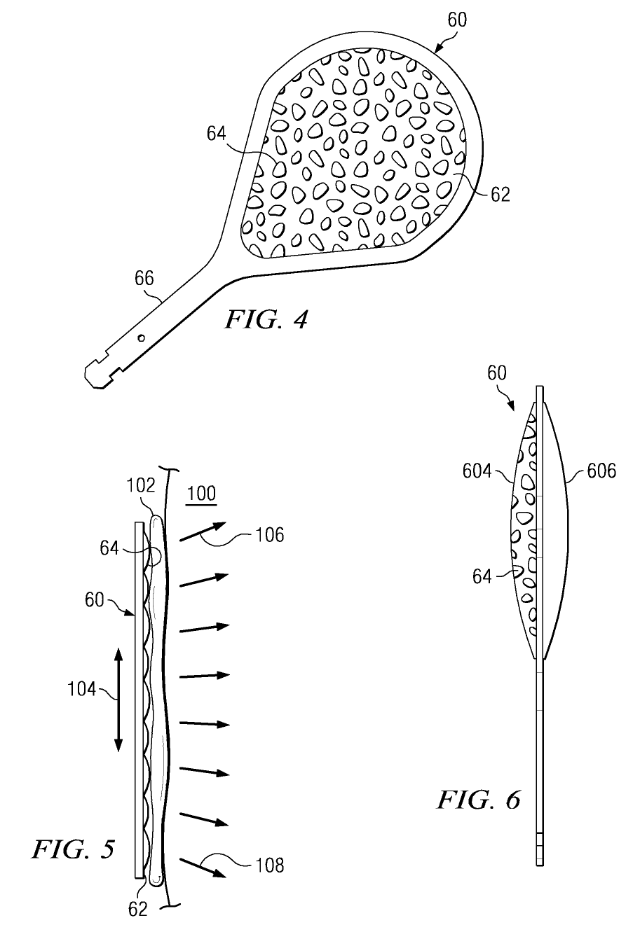

Referring now to FIG. 4, a paddle 60 adapted for infusion of select substances into the skin of a patient is shown. It may be seen that the paddle 60 includes a frontal surface 62 having a defined pattern 64 thereon. The pattern 64 may be varied depending on the particular applications. For example, a paddle design similar to the abrasive design, but of a different material may be used with effectiveness. This development is explained in more detail below. It may be seen that a stem 66 of the infusion paddle 60 is of a similar construction to that of paddles 40 and 42 in FIG. 2, so that paddle 60 may likewise be inserted into distal end 20 of wand 16 for application on a patient. The paddle 60 may be fabricated from polycarbonate, polyethylene, polypropylene or other similar material.

Reference is again specifically made to the above-referenced applications, the entirety of each being incorporated by reference. In accordance with the principles of the present invention, a select substance such as a lotion, cream, or other dermatological treatment ingredients (including anesthetics, and the like) are applied either to the face 50 or the paddle 60 and/or both and vibrationally infused into the skin of patient 52. An abrasive paddle may likewise be used for infusion. When the substance infusion follows the dermatological abrasion represented in FIG. 3, the skin surface 50 of the patient 52 is generally in condition for more effective absorption of the select substance by means of the kinetic application set forth herein and further described in the aforesaid applications.

Referring now to FIG. 5, there is shown an enlarged diagrammatical side-elevational view of the paddle 60 against the skin area 100 of a patient. It may be seen that a lotion or cream 102 is illustrated herein for vibrational infusion into the skin surface 100. The vibration or motion of the paddle 60 is represented by the arrow 104, and arrows 106 and 108 illustrate the infusion angles imparted by the vibrational action of the paddle 60.

FIG. 6 illustrates yet another embodiment of the present invention wherein a two sided paddle 60 is shown. One side 604 includes a permanent abrasive surface while the other side 606 includes a surface with the above described pattern 64 included thereon. The two sided paddle 60 may be bowed as shown in FIG. 6 or have a planar surface. In this way, a single paddle can be constructed with permanent abrasive and substantially non-abrasive surfaces for removing excess skin cells and substance infusion. One technique would be to fabricate the two sided paddle from structurally reinforced polycarbonate, polypropylene, and/or polyethylene to provide a surface on the one side for infusing a material into a patients skin and an ultrasonically welded abrasive etched, stainless steel surface on the other side for exfoliation.

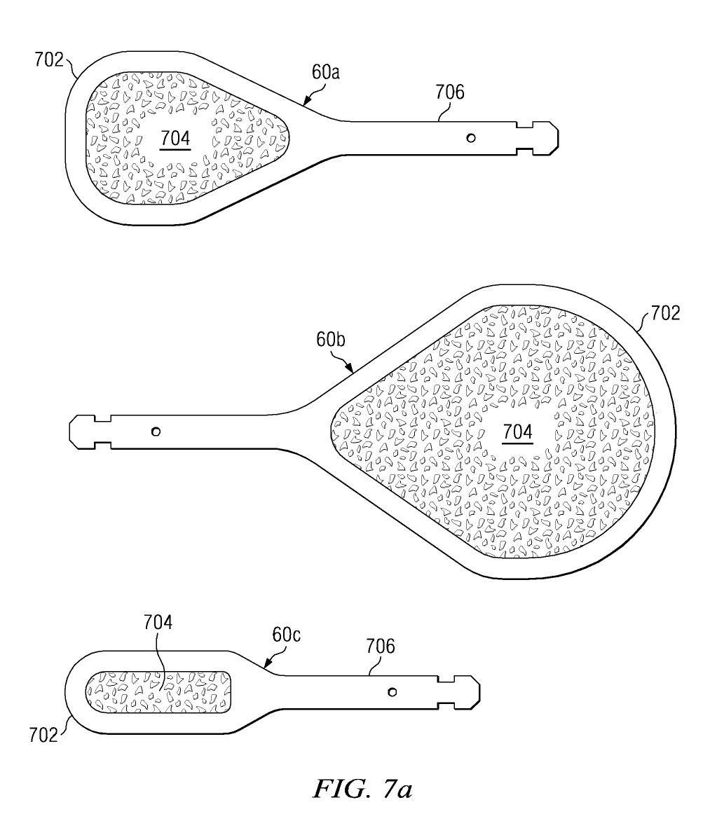

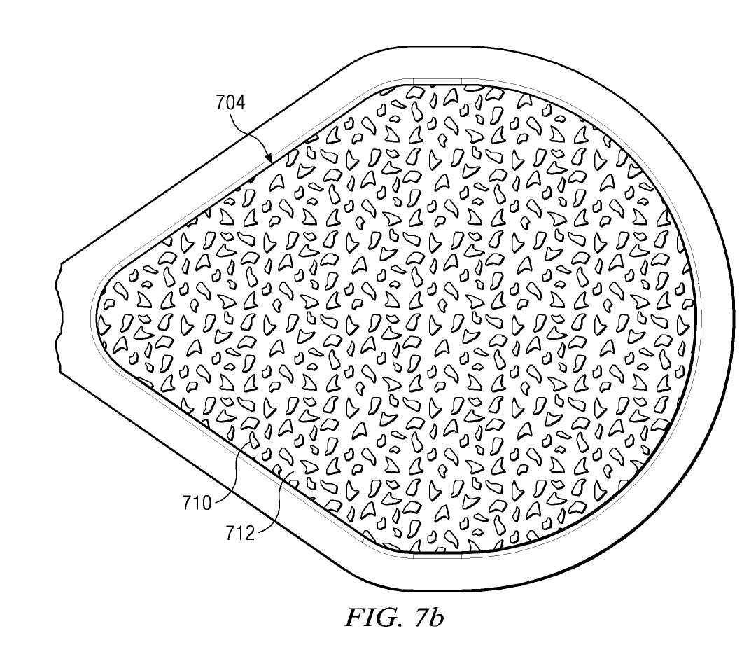

Referring now more particularly to FIG. 7a, there is illustrated three dual-sided treatment paddles 60. The three sizes of paddles have been found to be advantageous so that large and small areas of the human body may be treated separately. The paddle 60a comprises a medium sized paddle for treating intermediately sized skin surface areas. Paddle 60b comprises the largest paddle for treating large areas such as the back or chest of a patient. Paddle 60c comprises the smallest paddle for treating smaller skin areas such as a patient's face. The paddle 60a consists of a frame consisting of the paddle blade 702 onto which a metal surface 704 is placed. Alternatively, other types of material than metal may be used. The metal surface 704 comprises the abrasive surface which is used for exfoliating the skin of a patient. The paddle blade 702 is connected with the stem 706 which is integrated with the paddle blade 702 at a first end and contains a second end for being interconnected with a wand for imparting vibrational energies to a paddle.

Referring now to FIG. 7b, there is more closely illustrated the metal surface 704 comprising the abrasive surface. The metal insert 704 includes a number of raised protrusions 710 which are included in relatively flat open areas 712 surrounding each of the raised protrusions 710. The pattern of raised protrusions 710 in one embodiment is etched into the surface of the metal insert 704 into a pattern affording the abrasive surface necessary for exfoliation of a skin area in accordance with the present disclosure.

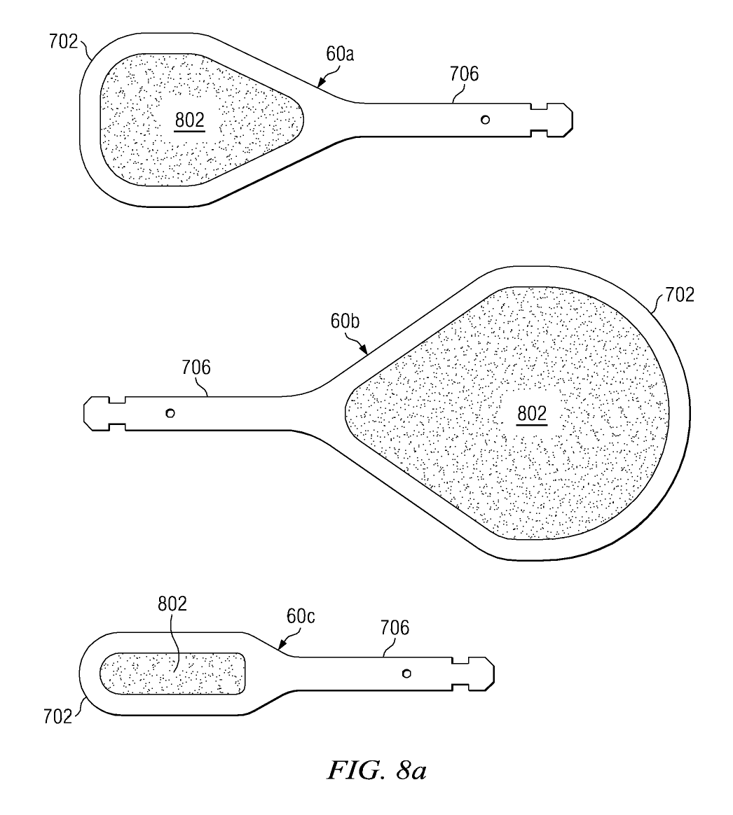

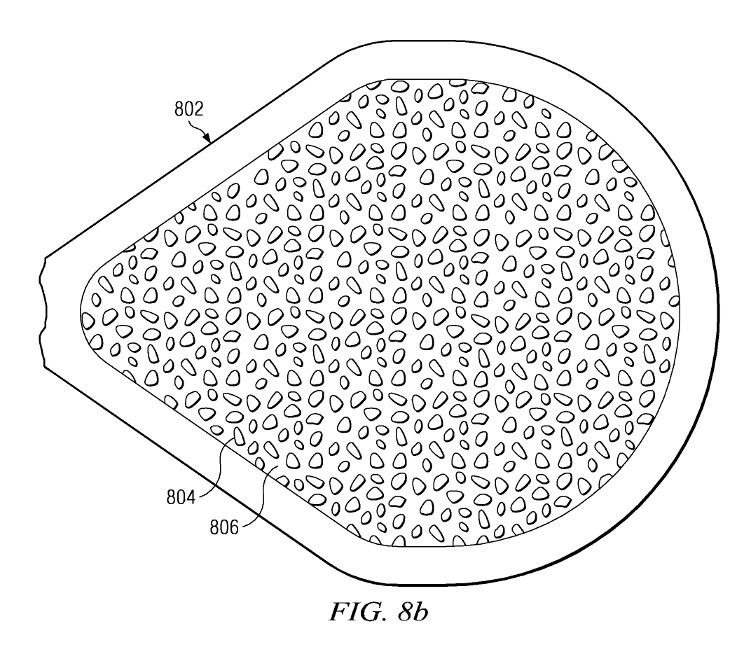

Referring now to FIG. 8a, there is illustrated a close-up view of the infusion side of the paddles 60. As before, there are paddles of three different sizes for infusion to large and smaller areas of the human body. The paddles 60 consist of a paddle blade 702 and the paddle stems 706 as described herein above. On each of the paddle blades 702 are affixed an infusion surface 802. The infusion surface 802 may, in one embodiment, be made of a plastic insert that has etched thereon a less abrasive surface than that used for the abrasive surface 704 of FIG. 7b. The infusion surface may be a separate plastic insert that is connected to the paddle blade 702 or may be a surface that is etched or molded into the surface of the paddle blade 702. Alternatively, other types of material than plastic may be used.

Referring now more particularly to FIG. 8b there is illustrated a close-up view of the infusion surface 802 of FIG. 8a. The infusion surface 802 consists of a number of protrusions 804 located within a flatter surface area 806. The protrusions 804 and the edges thereof are more rounded and less sharply angled than those of the abrasive surface 704 of FIG. 7 since the infusion surface is used for infusing a cream, lotion or other dermatological material into the skin of a patient rather than exfoliating the skin. The protrusions 804 of the infusion surface may be made by etching, molding, or other techniques useful for working with plastic material such as polyethylene, polypropylene, polycarbonate or similar materials. The infusion surface 802 does not contain the same pattern as that described in FIG. 4 but is a surface that has been found to be acceptable for the infusion of materials into the skin of a patient. An operation the dual-sided paddle 60 of the present invention is found to be of a variety of sizes as set forth in the figures above.

While the above descriptions of the abrasive surface 704 and the infusion surface 802 have been described using a metal material for the abrasive surface 704 and a plastic material for the infusion surface 802, other types of materials may be utilized for these surfaces. The necessary functionality of each of the surfaces is that the abrasive surface 704 be of a sufficiently rough texture to enable vibrational forces imparted to the paddle 60 to exfoliate the skin area of a patient. Likewise, the infusion surface 802 should have a less abrasive surface enabling the surface to perform the infusions processes of the paddle 60.

In a preferred embodiment, dual-sided paddles 60 of varying sizes are provided in a single array contained within a single package to enable the operator to select the most appropriate paddle for the dermatological abrasion and/or infusion and/or both. Since the paddles illustrated in the photographs have both an abrasive side and an infusion side, each paddle 60 is available for selection for the most appropriate dermatological treatment. Consistent with this availability is the capability of the operator to immediately provide dermatological infusion by utilizing the infusion side of the paddle 60 following use of the abrasive side of the paddle 60 for exfoliation. This allows the saving of time during the dermatological treatment when exfoliation and infusion are to be incorporated into the treatment modality for a given patient and select areas require varying sizes of treatment paddles. This aspect can be appreciated when one compares large open skin areas of the body such as the chest to the narrow areas of the human face near the eyes. By utilizing an array of paddles 60 having dual sides in accordance with the principals discussed herein, a great deal of time can be saved and efficiencies increased. In some instances, patient comfort can be further augmented with the present disclosure.

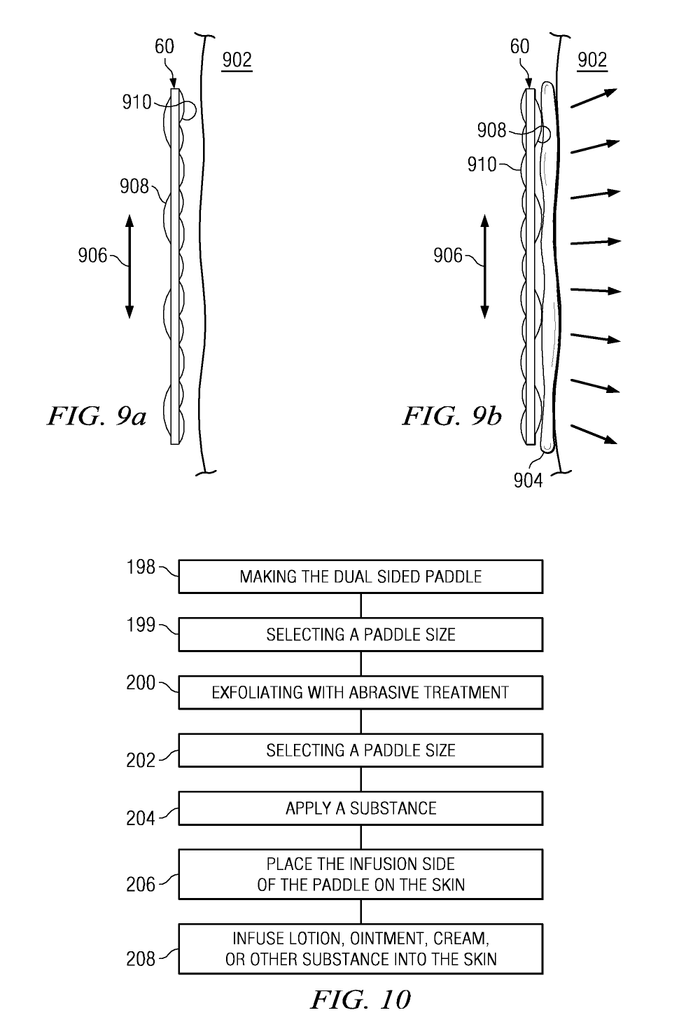

It may be further understood that when the system of the present disclosure is used by individual patients, the ability to use a dual-sided paddle for both abrasion and infusion will eliminate the need to remove the paddle 60 from the wand and for the sequential steps of exfoliation and infusion. This flexibility in manual operation can be psychologically conducive to use of the system due to the simplification of the overall operation. When an individual is using the dermatological system of the present invention in one's own home, it has been found to be inconvenient to manually change paddles on the wand when a separate paddle is needed for infusion relative to the exfoliation step. This psychological impediment for personal use can reduce the frequency of use of the system of the present disclosure by individuals who do not have available to them a third-party operator for the dermatological treatment. Yet the benefits can sill reside for an individual utilizing the system of the present disclosure in one's own home and the advantages of the system are appreciated when such ease in use and dual-sided treatment accommodation is provided. Likewise, when an individual is utilizing the paddles on a large surface that has been exfoliated, it is often desirable to immediately apply a lotion or cream or other topical substance into the exfoliated area for immediate infusing. This can be done without changing the paddle in the wand because the wand may simply be turned over to have available to the user the ability to have a substance infused immediately following exfoliation. Once infusion of one area is complete, the user is able to change the paddle when a smaller paddle is needed, for example, on the user's face. Similarly, the user does not have to stop and change out the paddle for the infusion step following exfoliation, because the user can simply rotate the wand to have it expose the infusion side. When the user simply applies cream or lotion to the exfoliated area, the wand can be used immediately and, in some instance, without even turning it off. This process is more fully illustrated in FIGS. 9a and 9b.

Referring now to FIGS. 9a and 9b, there is illustrated the manner in which each side of the dual-sided paddle 60 may be used as described herein above. The dual-sided paddle 60 utilizes the abrasive side 910 having the rougher surface thereon for initially exfoliating the skin 902 of the patient being treated. Once the exfoliation treatment has been provided by the abrasive side 910 of the dual-sided paddle 60, the patient may have a lotion, cream or other dermatological aid 904 applied to their skin 902, and the opposite infusion side 908 of the dual-sided paddle 60 is used to infuse the lotion, cream or other dermatological aid 904 into the skin of the patient. Both the exfoliation process illustrated in FIG. 9a and the infusion process illustrated in FIG. 9b are facilitated by the vibrations of the dual-sided paddle 60 as indicated generally by arrows 906.

Referring now to FIG. 10, there is illustrated a flow diagram describing one embodiment of the dermatological use of the dual-sided paddle described herein above. A dual-sided paddle is provided for use in the described procedure at step 198. Next, a paddle size is selected at step 199 for exfoliation of a patient's skin. The size of the paddle that is selected for exfoliation is based upon the area of skin to be exfoliated. Thus, a chest or back of an individual would require a larger paddle for exfoliating than would an area on the face of a patient. Next, at step 200, the area of skin that is to be treated is exfoliated by the abrasive side 910 of the dual-sided paddle 60 in order to expose an underlying layer of skin cells. Following exfoliation, a next paddle size is selected for infusion of a material into the skin that has been exfoliated at step 202. In many embodiments, the paddle size will be the same as that used for exfoliation and this will merely involve selecting the opposite side of the dual-sided paddle. A substance is applied to the skin of the patient or the paddle at step 204. The substance may comprise a cream, ointment, lotion or the like and/or other materials having dermatological ingredients adapted for skin treatment. The application of the substance may be upon the treated skin area, upon the dual-sided paddle, or both. The infusion side 908 of the dual-sided paddle 60 is then placed upon the skin of the patient at step 206. The lotion, ointment, cream or other substance is infused into the skin at step 208 using the vibrational movement of the paddle.

In summary, effective use of dermatological practitioners on patients is enhanced by the dual-sided paddles provided in the selected array of paddles described herein. Likewise, the personal use of the system of the present invention is further maximized for private use by facilitating a lone individual's use through ease and having both exfoliation and infusion within the same wand without a third party's intervention and without the user having to turn off the vibrational action of the wand prior to the second infusion step. Although it may not be immediately recognized to individuals not familiar with such dermatological treatment systems, ease of use in one's own home is a major consideration and further increases the utilitarian aspects of the present invention.

It will be appreciated by those skilled in the art having the benefit of this disclosure that this dual-sided paddle for use with vibrational energy under 1000 hertz for dermatological treatment provides an improved system and method for dermatological treatments. It should be understood that the drawings and detailed description herein are to be regarded in an illustrative rather than a restrictive manner, and are not intended to be limiting to the particular forms and examples disclosed. On the contrary, included are any further modifications, changes, rearrangements, substitutions, alternatives, design choices, and embodiments apparent to those of ordinary skill in the art, without departing from the spirit and scope hereof, as defined by the following claims. Thus, it is intended that the following claims be interpreted to embrace all such further modifications, changes, rearrangements, substitutions, alternatives, design choices, and embodiments.

What is claimed is:

1. A system for dermatological treatment of an individual's skin area, the system comprising:
a paddle defining on a first side thereof a first dermatological treatment surface made of at least one material having a sufficiently abrasive side for vibratory removal of an outer skin area of an individual, the paddle further defining on a second side thereof opposite the first side, a second dermatological treatment surface made of at least one material having a side specifically adapted for the vibratory infusion of a cream, lotion, or other dermatological aid into an underlying skin are of said individual;
a wand adapted to transfer a vibrational energy to said paddle such that the first and second dermatological treatment surfaces vibrate responsive to the vibrational energy; and
a control unit connected to said wand and further capable of generating a vibrational energy in said wand at a frequency of generally less than 1000 Hz.

2. The system of claim 1, wherein said control unit is capable of producing an adjustable frequency generally within the range of 0 and 1000 Hz.

3. The system of claim 1, wherein said first dermatological treatment surface contains a plurality of protrusions of a regular shape defined by a pattern of recessed grooves, said protrusions being specifically adapted for removal of the outer skin area.

4. The system of claim 1, wherein said second dermatological treatment surface contains a plurality of protrusions of an irregular shape defined by a plurality of recessed grooves, said protrusions being specifically adapted for vibratory infusion of said cream, lotion, or other dermatological aid.

5. The system of claim 1, wherein said second dermatological treatment surface has a substantially flat side on which the plurality of protrusions resides.

6. The system of claim 1, wherein said second dermatological treatment surface has a substantially curved surface on which the plurality of protrusions resides.

7. The system of claim 1, wherein said control unit is adapted to generate a vibrational energy in said wand on the order of 100 Hz.

8. The system of claim 1, wherein said control unit is adapted to generate a vibrational energy in said wand on the order of the range of 80-200 Hz.

9. The system of claim 1, wherein said first dermatological treatment surface is made of metal.

10. The system of claim 1, wherein said second dermatological treatment is made of plastic.

11. A treatment paddle for use with a system for dermatological treatment of an individual's skin area, the treatment paddle comprising:
a first dermatological treatment surface made of at least one material having a sufficiently abrasive side for vibratory removal of an outer skin area of an individual;
a second dermatological treatment surface made of at least one material located on an opposite side of the first dermatological treatment surface having a side specifically adapted for the vibratory infusion of a cream, lotion, or other dermatological aid into an underlying skin area of said individual; and
a stem defining a first and second ends, the first end of the stem integrated with the first and second dermatological surfaces and a second end of the stem adapted for connecting to a vibrational unit to receive vibrational energy therefrom.

12. The treatment paddle of claim 11, wherein said treatment paddle is vibrated at a frequency generally within the range of 0 and 1000 Hz.

13. The treatment paddle of claim 11, wherein said first dermatological treatment surface contains a plurality of protrusions of a regular shape defined by a pattern of recessed grooves, said protrusions being specifically adapted for removal of the outer skin area.

14. The treatment paddle of claim 11, wherein said second dermatological treatment surface contains a plurality of protrusions of an irregular shape defined by a plurality of recessed grooves, said protrusions being specifically adapted for vibratory infusion of said cream, lotion, or other dermatological aid.

15. The treatment paddle of claim 11, wherein said second dermatological treatment surface has a substantially flat side on which the plurality of protrusions resides.

16. The treatment paddle of claim 11, wherein said second dermatological treatment surface has a substantially curved surface on which the plurality of protrusions resides.

17. The treatment paddle of claim 11, wherein said treatment paddle is vibrated at a frequency on the order of 100 Hz.

18. The treatment paddle of claim 9, wherein said treatment paddle is vibrated at a frequency on the order of the range of 80-200 Hz.

19. The system of claim 11, wherein said first dermatological treatment surface is made of metal.

20. The system of claim 11, wherein said second dermatological treatment is made of plastic.
